(19) United States
(12) Patent Application Publication
(10) Pub. No.: US 2008/0132884 A1
(43) Pub. Date: Jun. 5, 2008

(54) Systems for treating tissue sites using electroporation

(76) Inventors: Boris Rubinsky, Albany, CA (US); Gary Onik, Orlando, FL (US); Paul Mikus, Coto de Cazz, CA (US)

Correspondence Address: HELLER EHRMAN LLP, 275 MIDDLEFIELD ROAD, MENLO PARK, CA, 94025-3506, US

(21) Appl. No.: 11/945,772

(22) Filed: Nov. 27, 2007

Related U.S. Application Data
(60) Provisional application No. 60/868,226, filed on Dec. 1, 2006.

(51) Int. Cl. A61B 18/12

(52) U.S. Cl. 606/34; 606/50

(57) ABSTRACT

A systems for treating a tissue site. At least first and second mono-polar electrodes are configured to be introduced at or near a tissue site of the patient. A voltage pulse generator is coupled to the first and second mono-polar electrodes. The voltage pulse generator is configured to apply sufficient electrical pulses between the first and second mono-polar electrodes to induce electroporation of cells in the tissue site, to create necrosis of cells of the tissue site, but insufficient to create a thermal damaging effect to a majority of the tissue site.

### CROSS-REFERENCE TO RELATED APPLICATIONS

This application claims the benefit of U.S. 60/868,226, filed Dec. 1, 2006, which application is fully incorporated herein by reference.

### BACKGROUND

1. Field of the Invention

This invention relates generally to electroporation, and more particularly to systems and methods for treating tissue sites of a patient using electroporation.

2. Description of the Related Art

Electroporation is defined as the phenomenon that makes cell membranes permeable by exposing them to certain electric pulses (Weaver, J. C. and Y. A. Chizmadzhev, Theory of electroporation: a review. Bioelectrochem. Bioenerg., 1996. 41: p. 135-60). The permeabilization of the membrane can be reversible or irreversible as a function of the electrical parameters used. In reversible electroporation the cell membrane reseals a certain time after the pulses cease and the cell survives. In irreversible electroporation the cell membrane does not reseal and the cell lyses. (Dev, S. B., Rabussay, D. P., Widera, G., Hofmann, G. A., Medical applications of electroporation, IEEE Transactions of Plasma Science, Vol 28 No 1, February 2000, pp 206-223).

Dielectric breakdown of the cell membrane due to an induced electric field, irreversible electroporation, was first observed in the early 1970s (Neumann, E. and K. Rosenheck, Permeability changes induced by electric impulses in vesicular membranes. J. Membrane Biol., 1972.10: p. 279-290; Crowley, J. M., Electrical breakdown of biomolecular lipid membranes as an electromechanical instability. Biophysical Journal, 1973.13: p. 711-724; Zimmermann, U., J. Vienken, and G. Pilwat, Dielectric breakdown of cell membranes, Biophysical Journal, 1974. 14(11): p. 881-899). The ability of the membrane to reseal, reversible electroporation, was discovered separately during the late 1970s (Kinosita Jr, K. and T. Y. Tsong, Hemolysis of human erythrocytes by a transient electric field. Proc. Natl. Acad. Sci. USA, 1977. 74(5): p. 1923-1927; Baker, P. F. and D. E. Knight, Calcium-dependent exocytosis in bovine adrenal medullary cells with leaky plasma membranes. Nature, 1978. 276: p. 620-622; Gauger, B. and F. W. Bentrup, A Study of Dielectric Membrane Breakdown in the Fucus Egg, J. Membrane Biol., 1979. 48(3): p. 249-264).

The mechanism of electroporation is not yet fully understood. It is thought that the electrical field changes the electrochemical potential around a cell membrane and induces instabilities in the polarized cell membrane lipid bilayer. The unstable membrane then alters its shape forming aqueous pathways that possibly are nano-scale pores through the membrane, hence the term “electroporation” (Chang, D. C., et al., Guide to Electroporation and Electrofusion. 1992, San Diego, Calif.: Academic Press, Inc.). Mass transfer can now occur through these channels under electrochemical control. Whatever the mechanism through which the cell membrane becomes permeabilized, electroporation has become an important method for enhanced mass transfer across the cell membrane.

The first important application of the cell membrane permeabilizing properties of electroporation is due to Neumann (Neumann, E., et al., Gene transfer into mouse lyoma cells by electroporation in high electric fields. J. EMBO, 1982.1: p. 841-5). He has shown that by applying reversible electroporation to cells it is possible to sufficiently permeabilize the cell membrane so that genes, which are macromolecules that normally are too large to enter cells, can after electroporation enter the cell. Using reversible electroporation electrical parameters is crucial to the success of the procedure, since the goal of the procedure is to have a viable cell that incorporates the gene.

Following this discovery electroporation became commonly used to reversible permeabilize the cell membrane for various applications in medicine and biotechnology to introduce into cells or to extract from cells chemical species that normally do not pass, or have difficulty passing across the cell membrane, from small molecules such as fluorescent dyes, drugs and radioactive tracers to high molecular weight molecules such as antibodies, enzymes, nucleic acids, HMW dextrans and DNA.

Following work on cells outside the body, reversible electroporation began to be used for permeabilization of cells in tissue. Heller, R., R. Gilbert, and M. J. Jaroszeski, Clinical applications of electrochemotherapy. Advanced drug delivery reviews, 1999. 35: p. 119-129. Tissue electroporation is now becoming an increasingly popular minimally invasive surgical technique for introducing small drugs and macromolecules into cells in specific areas of the body. This technique is accomplished by injecting drugs or macromolecules into the affected area and placing electrodes into or around the targeted tissue to generate reversible permeabilizing electric field in the tissue, thereby introducing the drugs or macromolecules into the cells of the affected area (Mir, L. M., Therapeutic perspectives of in vivo cell electropermeabilization. Bioelectrochemistry, 2001. 53: p. 1-10).

The use of electroporation to ablate undesirable tissue was introduced by Okino and Mohri in 1987 and Mir et al. in 1991. They have recognized that there are drugs for treatment of cancer, such as bleomycin and cys-platinum, which are very effective in ablation of cancer cells but have difficulties penetrating the cell membrane. Furthermore, some of these drugs, such as bleomycin, have the ability to selectively affect cancerous cells which reproduce without affecting normal cells that do not reproduce. Okino and Mori and Mir et al. separately discovered that combining the electric pulses with an impermeant anticancer drug greatly enhanced the effectiveness of the treatment with that drug (Okino, M. and H. Mohri, Effects of a high-voltage electrical impulse and an anticancer drug on in vivo growing tumors. Japanese Journal of Cancer Research, 1987. 78(12): p. 1319-21; Mir, L. M., et al., Electrochemotherapy potentiation of antitumour effect of bleomycin by local electric pulses. European Journal of Cancer, 1991. 27: p. 68-72). Mir et al. soon followed with clinical trials that have shown promising results and coined the treatment electrochemotherapy (Mir, L. M., et al., Electrochemotherapy, a novel antitumor treatment: first clinical trial C. R. Acad. Sci., 1991. Ser. III 313(613-8)).

Currently, the primary therapeutic in vivo applications of electroporation are antitumor electrochemotherapy (ECT), which combines a cytotoxic nonpermeant drug with permeabilizing electric pulses and electrogenetherapy (EGT) as a form of non-viral gene therapy, and transdermal drug delivery (Mir, L. M., Therapeutic perspectives of in vivo cell electropermeabilization. Bioelectrochemistry, 2001. 53: p. 1-10). The studies on electrochemotherapy and electrogenetherapy have been recently summarized in several publications (Jaroszeski, M. J., et al., In vivo gene delivery by electroporation. Advanced applications of electrochemistry, 1999. 35: p. 131-137; Heller, R., R. Gilbert, and M. J. Jaroszeski, Clinical applications of electrochemotherapy. Advanced drug delivery reviews, 1999. 35: p. 119-129; Mir, L. M., Therapeutic perspectives of in vivo cell electropermeabilization. Bioelectrochemistry, 2001. 53: p. 1-10; Davalos, R. V., Real Time Imaging for Molecular Medicine through electrical Impedance Tomography of Electroporation, in Mechanical Engineering. 2002, University of California at Berkeley: Berkeley. p. 237). A recent article summarized the results from clinical trials performed in five cancer research centers. Basal cell carcinoma, malignant melanoma, adenocarcinoma and head and neck squamous cell carcinoma were treated for a total of 291 tumors (Mir, L. M., et al., Effective treatment of cutaneous and subcutaneous malignant tumours by electrochemotherapy. British Journal of Cancer, 1998. 77(12): p. 2336-2342).

Electrochemotherapy is a promising minimally invasive surgical technique to locally ablate tissue and treat tumors regardless of their histological type with minimal adverse side effects and a high response rate (Dev, S. B., et al., Medical Applications of Electroporation. IEEE Transactions on Plasma Science, 2000. 28(1): p. 206-223; Heller, R., R. Gilbert, and M. J. Jaroszeski, Clinical applications of electrochemotherapy. Advanced drug delivery reviews, 1999. 35: p. 119-129). Electrochemotherapy, which is performed through the insertion of electrodes into the undesirable tissue, the injection of cytotoxic drugs in the tissue and the application of reversible electroporation parameters, benefits from the ease of application of both high temperature treatment therapies and non-selective chemical therapies and results in outcomes comparable of both high temperature therapies and non-selective chemical therapies.

Irreversible electroporation, the application of electrical pulses which induce irreversible electroporation in cells is also considered for tissue ablation (Davalos, R. V., Real Time Imaging for Molecular Medicine through electrical Impedance Tomography of Electroporation, in Mechanical Engineering. 2002, PhD Thesis, University of California at Berkeley: Berkeley, Davalos, R., L. Mir, Rubinsky B., “Tissue ablation with irreversible electroporation” in print February 2005 Annals of Biomedical Eng,). Irreversible electroporation has the potential for becoming and important minimally invasive surgical technique. However, when used deep in the body, as opposed to the outer surface or in the vicinity of the outer surface of the body, it has a drawback that is typical to all minimally invasive surgical techniques that occur deep in the body, it cannot be closely monitored and controlled. In order for irreversible electroporation to become a routine technique in tissue ablation, it needs to be controllable with immediate feedback. This is necessary to ensure that the targeted areas have been appropriately treated without affecting the surrounding tissue. This invention provides a solution to this problem in the form of medical imaging.

Medical imaging has become an essential aspect of minimally and non-invasive surgery since it was introduced in the early 1980's by the group of Onik and Rubinsky (G. Onik, C. Cooper, H. I. Goldenberg, A. A. Moss, B. Rubinsky, and M. Christianson, “Ultrasonic Characteristics of Frozen Liver,” Cryobiology, 21, pp. 321-328, 1984, J. C. Gilbert, G. M. Onik, W Haddick, and B. Rubinsky, “The Use of Ultrasound Imaging for Monitoring Cryosurgery,” Proceedings 6th Annual Conference, IEEE Engineering in Medicine and Biology, 107-112, 1984 G. Onik, J. Gilbert, WK. Haddick, R. A. Filly, P. W Collen, B. Rubinsky, and L. Farrel, “Sonographic Monitoring of Hepatic Cryosurgery, Experimental Animal Model,” American J. of Roentgenology, May 1985, pp. 1043-1047.) Medical imaging involves the production of a map of various physical properties of tissue, which the imaging technique uses to generate a distribution. For example, in using x-rays a map of the x-ray absorption characteristics of various tissues is produced, in ultrasound a map of the pressure wave reflection characteristics of the tissue is produced, in magnetic resonance imaging a map of proton density is produced, in light imaging a map of either photon scattering or absorption characteristics of tissue is produced, in electrical impedance tomography or induction impedance tomography or microwave tomography a map of electrical impedance is produced.

Minimally invasive surgery involves causing desirable changes in tissue, by minimally invasive means. Often minimally invasive surgery is used for the ablation of certain undesirable tissues by various means. For instance in cryosurgery the undesirable tissue is frozen, in radio-frequency ablation, focused ultrasound, electrical and micro-waves hyperthermia tissue is heated, in alcohol ablation proteins are denaturized, in laser ablation photons are delivered to elevate the energy of electrons. In order for imaging to detect and monitor the effects of minimally invasive surgery, these should produce changes in the physical properties that the imaging technique monitors.

The formation of nanopores in the cell membrane has the effect of changing the electrical impedance properties of the cell (Huang, Y, Rubinsky, B., “Micro-electroporation: improving the efficiency and understanding of electrical permeabilization of cells” Biomedical Microdevices, Vo 3, 145-150, 2000. (Discussed in “Nature Biotechnology” Vol 18. pp 368, April 2000), B. Rubinsky, Y Huang. “Controlled electroporation and mass transfer across cell membranes U.S. Pat. No. 6,300,108, Oct. 9, 2001).

Thereafter, electrical impedance tomography was developed, which is an imaging technique that maps the electrical properties of tissue. This concept was proven with experimental and analytical studies (Davalos, R. V., Rubinsky, B., Otten, D. M., “A feasibility study for electrical impedance tomography as a means to monitor tissue electroporation in molecular medicine” IEEE Trans of Biomedical Engineering. Vol. 49, No. 4 pp 400-404, 2002, B. Rubinsky, Y. Huang. “Electrical Impedance Tomography to control electroporation” U.S. Pat. No. 6,387,671, May 14, 2002.)

There is a need for improved systems and methods for treating a tissue site using electroporation.

### SUMMARY OF THE INVENTION

Accordingly, an object of the present invention is to provide improved systems and methods for treating tissue sites using electroporation.

Another object of the present invention is to provide systems and method for treating tissue sites using electroporation using sufficient electrical pulses to induce electroporation of cells in the tissue site, without creating a thermal damage effect to a majority of the tissue site.

Yet another object of the present invention is to provide systems and methods for treating tissue sites using electroporation with real time monitoring.

A further object of the present invention is to provide systems and methods for treating tissue sites using electroporation where the electroporation is performed in a controlled manner with monitoring of electrical impedance;

Still a further object of the present invention is to provide systems and methods for treating tissue sites using electroporation that is performed in a controlled manner, with controlled intensity and duration of voltage.

Another object of the present invention is to provide systems and methods for treating tissue sites using electroporation that is performed in a controlled manner, with a proper selection of voltage magnitude.

Yet another object of the present invention is to provide systems and methods for treating tissue sites using electroporation that is performed in a controlled manner, with a proper selection of voltage application time.

A further object of the present invention is to provide systems and methods for treating tissue sites using electroporation, and a monitoring electrode configured to measure a test voltage delivered to cells in the tissue site and remote sites such as the rectum and the urethra.

Still a further object of the present invention is to provide systems and methods for treating tissue sites using electroporation that is performed in a controlled manner to provide for controlled pore formation in cell membranes.

Still another object of the present invention is to provide systems and methods for treating tissue sites using electroporation that is performed in a controlled manner to create a tissue effect in the cells at the tissue site while preserving surrounding tissue.

Another object of the present invention is to provide systems and methods for treating tissue sites using electroporation, and detecting an onset of electroporation of cells at the tissue site.

Yet another object of the present invention is to provide systems and methods for treating tissue sites using electroporation where the electroporation is performed in a manner for modification and control of mass transfer across cell membranes.

A further object of the present invention is to provide systems and methods for treating tissue sites using electroporation, and an array of electrodes that creates a boundary around the tissue site to produce a volumetric cell necrosis region.

These and other objects of the present invention are achieved in, a system for treating a tissue site. At least first and second mono-polar electrodes are configured to be introduced at or near a tissue site of the patient. A voltage pulse generator is coupled to the first and second mono-polar electrodes. The voltage pulse generator is configured to apply sufficient electrical pulses between the first and second mono-polar electrodes to induce electroporation of cells in the tissue site, to create necrosis of cells of the tissue site, but insufficient to create a thermal damaging effect to a majority of the tissue site.

In another embodiment of the present invention, a system for treating a tissue site is provided. A bipolar electrode is configured to be introduced at or near a tissue site of the patient. A voltage pulse generator is coupled to the bipolar electrode. The voltage pulse generator is configured to apply sufficient electrical pulses to the bipolar electrode to induce electroporation of cells in the tissue site, to create necrosis of cells of the tissue site, but insufficient to create a thermal damaging effect to a majority of the tissue site.

In another embodiment of the present invention, a method is provided for treating A tissue site. At least first and second mono-polar electrodes are introduced to a tissue site of a patient. The at least first and second mono-polar electrodes are positioned at or near the tissue site. An electric field is applied in a controlled manner to the tissue site. The electric field is sufficient to produce electroporation of cells at the tissue site, and below an amount that causes thermal damage to a majority of the tissue site.

In another embodiment of the present invention, a method is provided for treating A tissue site. A bipolar electrode is introduced to a tissue site of a patient. The bipolar electrode is positioned at or near the tissue site. An electric field is applied in a controlled manner to the tissue site. The electric field is sufficient to produce electroporation of cells at the tissue site, and below an amount that causes thermal damage to a majority of the tissue site.

### BRIEF DESCRIPTION OF THE DRAWINGS

FIG. 1 illustrates a schematic diagram for one embodiment of a electroporation system of the present invention.

FIG. 2(a) illustrates an embodiment of the present invention with two mono-polar electrodes that can be utilized for electroporation with the FIG. 1 system.

FIG. 2(b) illustrates an embodiment of the present invention with three mono-polar electrodes that can be utilized for electroporation with the FIG. 1 system.

FIG. 2(c) illustrates an embodiment of the present invention with a single bi-polar electrode that can be utilized for electroporation with the FIG. 1 system.

FIG. 2(d) illustrates an embodiment of the present invention with an array of electrodes coupled to a template that can be utilized for electroporation with the FIG. 1 system.

FIG. 3 illustrates one embodiment of the present invention with an array of electrodes positioned around a tissue site, creating a boundary around the tissue site to produce a volumetric cell necrosis region.

### DETAILED DESCRIPTION

### Definitions

The term “reversible electroporation” encompasses permeabilization of a cell membrane through the application of electrical pulses across the cell. In “reversible electroporation” the permeabilization of the cell membrane ceases after the application of the pulse and the cell membrane permeability reverts to normal or at least to a level such that the cell is viable. Thus, the cell survives “reversible electroporation.” It may be used as a means for introducing chemicals, DNA, or other materials into cells.

The term “irreversible electroporation” also encompasses the permeabilization of a cell membrane through the application of electrical pulses across the cell. However, in “irreversible electroporation” the permeabilization of the cell membrane does not cease after the application of the pulse and the cell membrane permeability does not revert to normal and as such cell is not viable. Thus, the cell does not survive “irreversible electroporation” and the cell death is caused by the disruption of the cell membrane and not merely by internal perturbation of cellular components. Openings in the cell membrane are created and/or expanded in size resulting in a fatal disruption in the normal controlled flow of material across the cell membrane. The cell membrane is highly specialized in its ability to regulate what leaves and enters the cell. Irreversible electroporation destroys that ability to regulate in a manner such that the cell can not compensate and as such the cell dies.

“Ultrasound” is a method used to image tissue in which pressure waves are sent into the tissue using a piezoelectric crystal. The resulting returning waves caused by tissue reflection are transformed into an image.

“MRI” is an imaging modality that uses the perturbation of hydrogen molecules caused by a radio pulse to create an image.

“CT” is an imaging modality that uses the attenuation of an x-ray beam to create an image.

“Light imaging” is an imaging method in which electromagnetic waves with frequencies in the range of visible to far infrared are send into tissue and the tissue's reflection and/or absorption characteristics are reconstructed.

“Electrical impedance tomography” is an imaging technique in which a tissue's electrical impedance characteristics are reconstructed by applying a current across the tissue and measuring electrical currents and potentials

In accordance with the present invention specific imaging technologies used in the field of medicine are used to create images of tissue affected by electroporation pulses. The images are created during the process of carrying out irreversible electroporation and are used to focus the electroporation on tissue to be ablated and to avoid ablating tissue such as nerves. The process of the invention may be carried out by placing electrodes, such as a needle electrode in the imaging path of an imaging device. When the electrodes are activated the image device creates an image of tissue being subjected to electroporation. The effectiveness and extent of the electroporation over a given area of tissue can be determined in real time using the imaging technology.

Reversible electroporation requires electrical parameters in a precise range of values that induce only reversible electroporation. To accomplish this precise and relatively narrow range of values (between the onset of electroporation and the onset of irreversible electroporation) when reversible electroporation devices are designed they are designed to generally operate in pairs or in a precisely controlled configuration that allows delivery of these precise pulses limited by certain upper and lower values. In contrast, in irreversible electroporation the limit is more focused on the lower value of the pulse which should be high enough to induce irreversible electroporation.

Higher values can be used provided they do not induce burning. Therefore the design principles are such that no matter how many electrodes are use the only constrain is that the electrical parameters between the most distant ones be at least the value of irreversible electroporation. If within the electroporated regions and within electrodes there are higher gradients this does not diminish the effectiveness of the probe. From these principles we can use a very effective design in which any irregular region to be ablated can be treated by surrounding the region with ground electrodes and providing the electrical pulses from a central electrode. The use of the ground electrodes around the treated area has another potential value—it protects the tissue outside the area that is intended to be treated from electrical currents and is an important safety measure. In principle, to further protect an area of tissue from stray currents it would be possible to put two layers of ground electrodes around the area to be ablated. It should be emphasized that the electrodes can be infinitely long and can also be curves to better hug the undesirable area to be ablated.

In one embodiment of the present invention, methods are provided to apply an electrical pulse or pulses to tissue sites. The pulses are applied between electrodes and are applied in numbers with currents so as to result in irreversible electroporation of the cells without damaging surrounding cells. Energy waves are emitted from an imaging device such that the energy waves of the imaging device pass through the area positioned between the electrodes and the irreversible electroporation of the cells effects the energy waves of the imaging device in a manner so as to create an image.

Typical values for pulse length for irreversible electroporation are in a range of from about 5 microseconds to about 62,000 milliseconds or about 75 microseconds to about 20,000 milliseconds or about 100 microseconds ±10 microseconds. This is significantly longer than the pulse length generally used in intracellular (nano-seconds) electro-manipulation which is 1 microsecond or less—see published U.S. application 2002/0010491 published Jan. 24, 2002. Pulse lengths can be adjusted based on the real time imaging.

The pulse is at voltage of about 100 V/cm to 7,000 V/cm or 200 V/cm to 2000 V/cn or 300V/cm to 1000 V/cm about 600 V/cm ±10% for irreversible electroporation. This is substantially lower than that used for intracellular electro-manipulation which is about 10,000 V/cm, see U.S. application 2002/0010491 published Jan. 24, 2002. The voltage can be adjusted alone or with the pulse length based on real time imaging information.

The voltage expressed above is the voltage gradient (voltage per centimeter). The electrodes may be different shapes and sizes and be positioned at different distances from each other. The shape may be circular, oval, square, rectangular or irregular etc. The distance of one electrode to another may be 0.5 to 10 cm., 1 to 5 cm., or 2-3 cm. The electrode may have a surface area of 0.1-5 sq. cm. or 1-2 sq. cm.

The size, shape and distances of the electrodes can vary and such can change the voltage and pulse duration used and can be adjusted based on imaging information. Those skilled in the art will adjust the parameters in accordance with this disclosure and imaging to obtain the desired degree of electroporation and avoid thermal damage to surrounding cells.

Thermal effects require electrical pulses that are substantially longer from those used in irreversible electroporation (Davalos, R. V., B. Rubinsky, and L. M. Mir, Theoretical analysis of the thermal effects during in vivo tissue electroporation. Bioelectrochemistry, 2003. Vol 61(1-2): p. 99-107). When using irreversible electroporation for tissue ablation, there may be concern that the irreversible electroporation pulses will be as large as to cause thermal damaging effects to the surrounding tissue and the extent of the tissue site ablated by irreversible electroporation will not be significant relative to that ablated by thermal effects. Under such circumstances irreversible electroporation could not be considered as an effective tissue site ablation modality as it will act in superposition with thermal ablation. To a degree, this problem is addressed via the present invention using imaging technology.

In one aspect of the invention the imaging device is any medical imaging device including ultrasound, X-ray technologies, magnetic resonance imaging (MRI), light imaging, electrical impedance tomography, electrical induction impedance tomography and microwave tomography. It is possible to use combinations of different imaging technologies at different points in the process.

For example, one type of imaging technology can be used to precisely locate a tissue site, a second type of imaging technology can be used to confirm the placement of electrodes relative to the tissue site. And yet another type of imaging technology could be used to create images of the currents of irreversible electroporation in real time. Thus, for example, MRI technology could be used to precisely locate the tissue site. Electrodes could be placed and identified as being well positioned using X-ray imaging technologies. Current could be applied to carry out irreversible electroporation while using ultrasound technology to determine the extent of tissue site effected by the electroporation pulses. It has been found that within the resolution of calculations and imaging the extent of the image created on ultrasound corresponds to an area calculated to be irreversibly electroporated. Within the resolution of histology the image created by the ultrasound image corresponds to the extent of tissue site ablated as examined histologically.

Because the effectiveness of the irreversible electroporation can be immediately verified with the imaging it is possible to limit the amount of unwanted damage to surrounding tissues and limit the amount of electroporation that is carried out. Further, by using the imaging technology it is possible to reposition the electrodes during the process. The electrode repositioning may be carried out once, twice or a plurality of times as needed in order to obtain the desired degree of irreversible electroporation on the desired tissue site.

In accordance with one embodiment of the present invention, a method may be carried out which comprises several steps. In a first step an area of tissue site to be treated by irreversible electroporation is imaged. Electrodes are then placed in the tissue site with the tissue site to be ablated being positioned between the electrodes. Imaging can also be carried out at this point to confirm that the electrodes are properly placed. After the electrodes are properly placed pulses of current are run between the two electrodes and the pulsing current is designed so as to minimize damage to surrounding tissue and achieve the desired irreversible electroporation of the tissue site. While the irreversible electroporation is being carried out imaging technology, is used and that imaging technology images the irreversible electroporation occurring in real time. While this is occurring the amount of current and number of pulses may be adjusted so as to achieve the desired degree of electroporation. Further, one or more of the electrodes may be repositioned so as to make it possible to target the irreversible electroporation and ablate the desired tissue site.

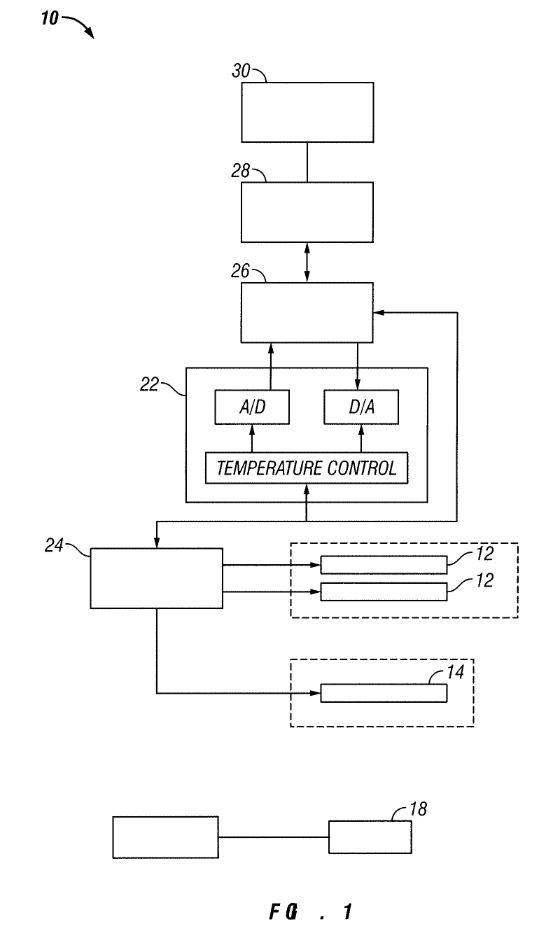

Referring to FIG. 1, one embodiment of the present invention provides a system, generally denoted as 10, for treating a tissue site of a patient.

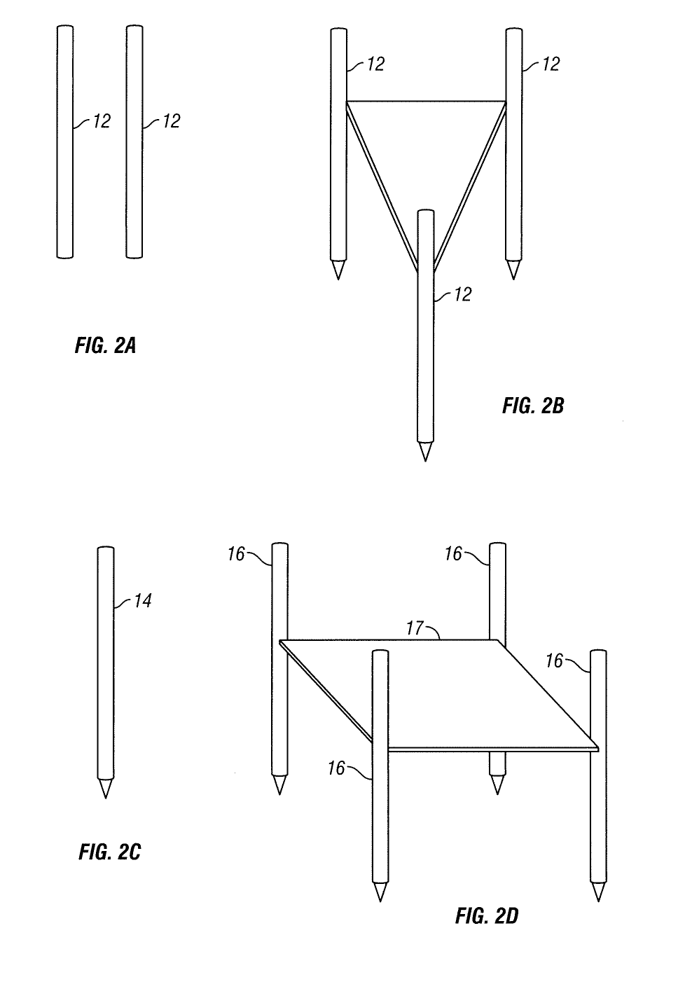

Two or more monopolar electrodes 12, one or more bipolar electrodes 14 or an array 16 of electrodes can be utilized, as illustrated in FIGS. 2(a)-2(d). In one embodiment, at least first and second monopolar electrodes 12 are configured to be introduced at or near the tissue site of the patient. It will be appreciated that three or more monopolar electrodes 12 can be utilized. The array 16 of electrodes is configured to be in a substantially surrounding relationship to the tissue site. The array 16 of electrodes can employ a template 17 to position and/or retain each of the electrodes. Template 17 can maintain a geometry of the array 16 of electrodes. Electrode placement and depth can be determined by the physician. The monopolar and bi-polar electrodes 12 and 14, and the array 16 of electrodes can be introduced through, the rectal wall, the peritoneum, urethra and the like.

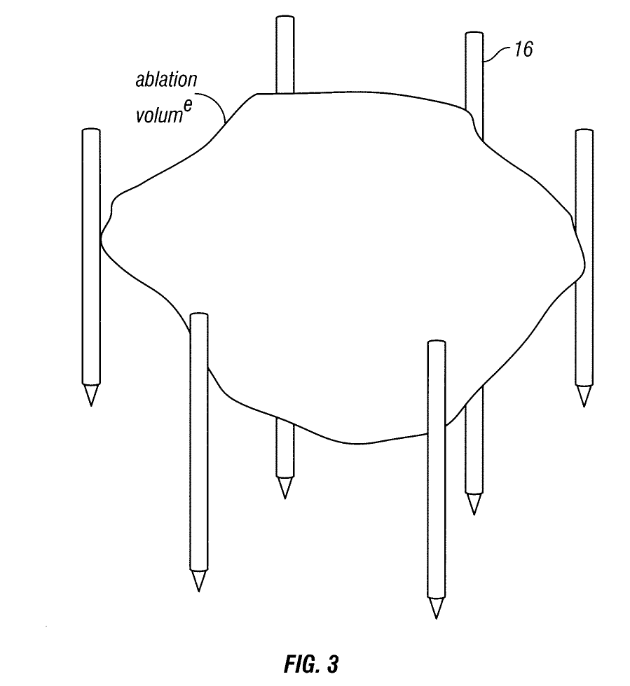

As shown in FIG. 3, the array 16 of electrodes creates a boundary around the tissue site to produce a volumetric cell necrosis region. Essentially, the array 16 of electrodes makes a treatment area the extends from the array 16 of electrodes, and extends in an inward direction. The array 16 of electrodes can have a pre-determined geometry, and each of the associated electrodes can be deployed individually or simultaneously at the tissue site either percutaneously, or planted in-situ in the patient.

In one embodiment, the monopolar electrodes 12 are separated by a distance of about 5 mm to 10 cm and they have a circular cross-sectional geometry. One or more additional probes 18 can be provided, including monitoring probes, an aspiration probe such as one used for liposuction, fluid introduction probes, and the like. Each bipolar electrode 14 can have multiple electrode bands 20. The spacing and the thickness of the electrode bands 20 is selected to optimize the shape of the electric field. In one embodiment, the spacing is about 1 mm to 5 cm typically, and the thickness of the electrode bands 20 can be from 0.5 mm to 5 cm.

Referring again to FIG. 1, a voltage pulse generator 22 is coupled to the electrodes 12, 14 and the array 16. The voltage pulse generator 22 is configured to apply sufficient electrical pulses between the first and second monopolar electrodes 12, bi-polar electrode 14 and array 16 to induce electroporation of cells in the tissue site, and create necrosis of cells of the tissue site. However, the applied electrical pulses are insufficient to create a thermal damaging effect to a majority of the tissue site.

The electrodes 12, 14 and array 16 are each connected through cables to the voltage pulse generator 22. A switching device 24 can be included. The switching device 24, with software, provides for simultaneous or individual activation of multiple electrodes 12, 14 and array 16. The switching device 24 is coupled to the voltage pulse generator 22. In one embodiment, means are provided for individually activating the electrodes 12, 14 and array 16 in order to produce electric fields that are produced between pre-selected electrodes 12, 14 and array 16 in a selected pattern relative to the tissue site. The switching of electrical signals between the individual electrodes 12, 14 and array 16 can be accomplished by a variety of different means including but not limited to, manually, mechanically, electrically, with a circuit controlled by a programmed digital computer, and the like. In one embodiment, each individual electrode 12, 14 and array 16 is individually controlled.

The pulses are applied for a duration and magnitude in order to permanently disrupt the cell membranes of cells at the tissue site. A ratio of electric current through cells at the tissue site to voltage across the cells can be detected, and a magnitude of applied voltage to the tissue site is then adjusted in accordance with changes in the ratio of current to voltage.

In one embodiment, an onset of electroporation of cells at the tissue site is detected by measuring the current. In another embodiment, monitoring the effects of electroporation on cell membranes of cells at the tissue site are monitored. The monitoring can be preformed by image monitoring using ultrasound, CT scan, MRI, CT scan, and the like.

In other embodiments, the monitoring is achieved using a monitoring electrode 18. In one embodiment, the monitoring electrode 18 is a high impedance needle that can be utilized to prevent preferential current flow to a monitoring needle. The high impedance needle is positioned adjacent to or in the tissue site, at a critical location. This is similar in concept and positioning as that of placing a thermocouple as in a thermal monitoring. Prior to the full electroporation pulse being delivered a “test pulse” is delivered that is some fraction of the proposed full electroporation pulse, which can be, by way of illustration and without limitation, 10%, and the like. This test pulse is preferably in a range that does not cause irreversible electroporation.

The monitoring electrode 18 measures the test voltage at the location. The voltage measured is then extrapolated back to what would be seen by the monitoring electrode 18 during the full pulse, e.g., multiplied by 10 in one embodiment, because the relationship is linear). If monitoring for a potential complication at the tissue site, a voltage extrapolation that falls under the known level of irreversible electroporation indicates that the tissue site where monitoring is taking place is safe. If monitoring at that tissue site for adequacy of electroporation, the extrapolation falls above the known level of voltage adequate for irreversible tissue electroporation.

In one embodiment in which the bipolar electrode 14 is placed transrectally the monitoring electrode 18 is integral to the bipolar electrode 14 placed either distal or proximal to the active bipolar electrodes 14. The monitoring electrode 18 is a fixed distance form the bipolar electrode 14. In another embodiment the monitoring electrode 18 is mounted on a sheath through which the bipolar electrode 14 is placed. The distance from the bipolar electrode 14 can then be varied and positioned based on imaging and the structure to be monitored, such as the rectal mucosa. In another embodiment the monitoring electrode 18 is mounted on a biopsy guide through which the bipolar electrode 14 is placed. The moniroing electrode 18 is placed at the tip of the guide and rests against the rectal mucosa as the bipolar electrode 14 is placed.

The effects of electroporation on cell membranes of cells at the tissue site can be detected by measuring the current flow.

In various embodiments, the electroporation is performed in a controlled manner, with real time monitoring, to provide for controlled pore formation in cell membranes of cells at the tissue site, to create a tissue effect in the cells at the tissue site while preserving surrounding tissue, with monitoring of electrical impedance, and the like.

The electroporation can be performed in a controlled manner by controlling the intensity and duration of the applied voltage and with or without real time control. Additionally, the electroporation is performed in a manner to provide for modification and control of mass transfer across cell membranes. Performance of the electroporation in the controlled manner can be achieved by selection of a proper selection of voltage magnitude, proper selection of voltage application time, and the like.

The system 10 can include a control board 26 that functions to control temperature of the tissue site. In one embodiment of the present invention, the control board 26 receives its program from a controller. Programming can be in computer languages such as C or BASIC (registered trade mark) if a personnel computer is used for a controller 28 or assembly language if a microprocessor is used for the controller 28. A user specified control of temperature can be programmed in the controller 28.

The controller 28 can include a computer, a digital or analog processing apparatus, programmable logic array, a hardwired logic circuit, an application specific integrated circuit (“ASIC”), or other suitable device. In one embodiment, the controller 28 includes a microprocessor accompanied by appropriate RAM and ROM modules, as desired. The controller 28 can be coupled to a user interface 30 for exchanging data with a user. The user can operate the user interface 30 to input a desired pulsing pattern and corresponding temperature profile to be applied to the electrodes 12, 14 and array 16.

By way of illustration, the user interface 30 can include an alphanumeric keypad, touch screen, computer mouse, push-buttons and/or toggle switches, or another suitable component to receive input from a human user. The user interface 30 can also include a CRT screen, LED screen, LCD screen, liquid crystal display, printer, display panel, audio speaker, or another suitable component to convey data to a human user. The control board 26 can function to receive controller input and can be driven by the voltage pulse generator 22.

In various embodiment, the voltage pulse generator 22 is configured to provide that each pulse is applied for a duration of about, 5 microseconds to about 62 seconds, 90 to 110 microseconds, 100 microseconds, and the like. A variety of different number of pulses can be applied, including but not limited to, from about 1 to 15 pulses, about eight pulses of about 100 microseconds each in duration, and the like. In one embodiment, the pulses are applied to produce a voltage gradient at the tissue site in a range of from about 50 volt/cm to about 8000 volt/cm.

In various embodiments, the tissue site is monitored and the pulses are adjusted to maintain a temperature of, 100 degrees C. or less at the tissue site, 75 degrees C. or less at the tissue site, 60 degrees C. or less at the tissue site, 50 degrees C. or less at the tissue site, and the like. The temperature is controlled in order to minimize the occurrence of a thermal effect to the tissue site. These temperatures can be controlled by adjusting the current-to-voltage ratio based on temperature.

In one embodiment of the present invention, the system 10 is utilized to treat a tissue site with electroporation of cells at a tissue site, creating cell necrosis in the tissue site around the urethra. The system 10 delivers electroporation pulses along the muscular fibers and nerves at the tissue site and produces a volume of necrotic cells at the tissue site around the urethra. Destruction of these nerves, that create an elevation in tension of the muscle fibers, is also achieved. The resulting necrotic tissue is removed by macrophages.

First and second mono-polar electrodes 12, or more, the bi-polar electrode 14 or the array 16 of electrodes are introduced through the rectal wall, the peritoneum or the urethra of the patient. The electroporation is positioned and monitored by image monitoring with ultrasound, CT scan, MRI, CT scan, and the like, or with a monitoring electrode 18. Each of the electrodes 12, 14 or array 16 can have insulated portions and is connected to the voltage pulse generator 22.

### EXAMPLE 1

An area of the tissue site is imaged. Two bi-polar electrodes 12, with sharpened distal ends, are introduced into in the tissue site through the rectal wall of the patient. The area of the tissue site to be ablated is positioned between the two electrodes. Imaging is used to confirm that the mono-polar electrodes are properly placed. The two mono-polar electrodes are separated by a distance of 5 mm to 10 cm at various locations of the tissue site. Pulses are applied with a duration of 5 microseconds to about 62 seconds each. Monitoring is preformed using ultrasound. The tissue site is monitored. In response to the monitoring, pulses are adjusted to maintain a temperature of no more than 100 degrees C. A voltage gradient at the tissue site in a range of from about 50 volt/cm to about 1000 volt/cm is created. A volume of the tissue site of about 1 cm by 0.5 cm undergoes cell necrosis.

### EXAMPLE 2

An area of the tissue site is imaged. Two mono-polar electrodes 12, are introduced into in the tissue site through the urethra of the patient. The area of the tissue site to be ablated is positioned between the two mono-polar electrodes 12. Imaging is used to confirm that the electrodes are properly placed. The two mono-polar electrodes 12 are separated by a distance of 5 mm to 10 cm at various locations of the tissue site. Pulses are applied with a duration of about 90 to 110 microseconds each.

Monitoring is performed using a CT scan. The tissue site is monitored. In response to the monitoring, pulses are adjusted to maintain a temperature of no more than 75 degrees C. A voltage gradient at the tissue site in a range of from about 50 volt/cm to about 5000 volt/cm is created. The tissue site undergoes cell necrosis.

### EXAMPLE 3

An area of the tissue site is imaged. The array 16 of electrodes are introduced into in the tissue site through the peritoneum of the patient. The array 16 of electrodes is positioned in a surrounding relationship to the tissue site. Imaging is used to confirm that the electrodes are properly placed. Pulses are applied with a duration of about 100 microseconds each. A monitoring electrode 18 is utilized. Prior to the full electroporation pulse being delivered a test pulse is delivered that is about 10% of the proposed full electroporation pulse. The test pulse does not cause irreversible electroporation. The tissue site is monitored. In response to the monitoring, pulses are adjusted to maintain a temperature of no more than 60 degrees C. A voltage gradient at the tissue site in a range of from about 50 volt/cm to about 8000 volt/cm is created. The tissue site undergoes cell necrosis.

### EXAMPLE 4

An area of the tissue site is imaged. A single bi-polar electrode 14, with a sharpened distal end, is introduced into the tissue site through the rectal wall of the patient. A monitoring electrode 18 is placed at a tip of a biopsy guide and rests against the rectal mucosa when the bipolar electrode 14 is placed. Imaging is used to confirm that the bi-polar electrode 14 is properly placed. Pulses are applied with a duration of 5 microseconds to about 62 seconds each. Monitoring is preformed using ultrasound. The tissue site is monitored. In response to the monitoring, pulses are adjusted to maintain a temperature of no more than 100 degrees C. A voltage gradient at the tissue site in a range of from about 50 volt/cm to about 1000 volt/cm is created. The tissue site undergoes cell necrosis.

### EXAMPLE 5

An area of the tissue site is imaged. A array 16 of electrodes is introduced into the tissue site through the rectal wall of the patient, and are positioned around the tissue site. Imaging is used to confirm that the array 16 of electrodes is properly placed. Pulses are applied with a duration of about 90 to 110 microseconds each. Monitoring is performed using a CT scan. The tissue site is monitored. In response to the monitoring, pulses are adjusted to maintain a temperature of no more than 75 degrees C. A voltage gradient at the tissue site in a range of from about 50 volt/cm to about 5000 volt/cm is created. The tissue site undergoes cell necrosis.

### EXAMPLE 6

An area of the tissue site is imaged. The array 16 of electrodes is introduced into the tissue site through the peritoneum of the patient, and positioned in a surrounding relationship to the tissue site. Imaging is used to confirm that the array 16 of electrodes is properly placed. Pulses are applied with a duration of about 100 microseconds each. A monitoring electrode 18 is utilized. Prior to the full electroporation pulse being delivered a test pulse is delivered that is about 10% of the proposed full electroporation pulse. The test pulse does not cause irreversible electroporation. The tissue site is monitored. In response to the monitoring, pulses are adjusted to maintain a temperature of no more than 60 degrees C. A voltage gradient at the tissue site in a range of from about 50 volt/cm to about 8000 volt/cm is created. The tissue site undergoes cell necrosis.

### EXAMPLE 7

### Arc Prevention During Irreversible Electroporation

During the application of electric pulses by electrodes to create sufficient field strength to cause irreversible electroporation, voltage gradients can get to sufficient amplitude that they may cause arcing to occur. The presence of an arc during certain clinical applications, particularly open surgery may not create a clinical issue. However during minimally invasive procedures, where the application of the electric pulses may be performed in tight spaces or near critical structures, arcing mat result in undesired clinical outcomes.

In various embodiments of the present invention, several methods are provided for reducing or eliminating the potential for arcing to occur during the application of electric pulses for irreversible electroporation. The following are given for purposes of illustration and do not limit the scope of the present invention.

### Method 1

At the interface of the conducting electrode (anode or cathode in bipolar configuration, active electrode in monopolar configuration) and the insulation, rounding the edges of the electrode and the insulation to cause a more gradual slope of voltage gradient. Either one or both of the edges may be rounded of shaped to cause this change in the voltage gradient at the interface.

### Method 2

Delivery of an electric pulse of sufficient amplitude to cause irreversible electroporation with a leading edge that ramps a rate that avoids the formation of a pressure wave that results in an arc at the delivery electrode. The rate and duration of the ramp of the leading edge can be varied to accommodate the specific clinical application.

### Method 3

A vacuum or suction cannula central or integrated into the delivery electrode of an irreversible electroporation pulse to cause relief of the development of potential arcs. This vacuum or suction could be drawn from the tip of the electrode or at various points of the delivery electrode that have a higher potential of arcing.

### Method 4

A vibrating signal applied to the shaft of the electrode sufficient to interfere with the development of gas bubbles that form prior to arcing. The vibrating signal is of sufficient strength and frequency to dislodge or interrupt the bubble formation.

### EXAMPLE 8

### Monitoring of IRE Using a Non-Electroporating Pre-Pulse with a High Impedance Needle Electrode

IRE is a new non-thermal ablation modality that uses a short micro second to millisecond pulse of DC current to ablate tissue. Since the ablation occurs virtually instantaneously monitoring of the ablation in the traditional manner with imaging or thermocouples over minutes is not possible. The proposed invention allows prediction of the safety and efficacy of an electroporating pulse before it is delivered in the tissue environment.

In one embodiment of the present invention, a separate monitoring needle is placed into the tissue to be monitored. The needle can either be placed into an area in which the tissue needs to be adequately ablated or in area of tissue that should not be ablated to due safety concerns.

An important aspect of this embodiment of the present invention is the use of a high impedance circuit associated with the monitoring electrode. This prevents the monitoring electrode from acting as an active ground electrode, drawing current to it and therefore giving false readings. Another embodiment of the invention includes the use of two electrodes to calculate an actual voltage gradient from two separate points in the tissue.

### EXAMPLE 9

### Use of Magnetic Fields to Ensure Catheter Contact in Intravascular Applications

Intimate contact of the catheter based electrodes to the endocardial wall is critical for successful and reproducible lesioning associated with arrhythmia ablation. The problem is compounded by cardiac motion, the intrinsic shape of the catheter, and the open space in the cardiac chambers.

In one embodiment of the present invention, the use of a catheter based ablation device that has a ferromagnetic portion is in the region of the ablation electrodes or other ablation affector, including but not limited to a microwave antenna, and the like. A magnet can then be placed outside the patients chest to attract the tip of the catheter to the correct location. The magnet then keeps the catheter tip against the endocardium despite the movements of the heart.

In one embodiment the tip of the catheter is actually a small electromagnet. This allows obviates the need for a ferromagnetic material on the catheter and allows control of the process from the catheter. In another embodiment the Magnet outside the patient can be an electromagnet which can be further controlled by applying current when needed. In other embodiments the outside magnet could be focused to a narrow field in order to direct the catheter from outside the patient.

The foregoing description of embodiments of the present invention has been presented for purposes of illustration and description. It is not intended to be exhaustive or to limit the invention to the precise forms disclosed. Obviously, many modifications and variations will be apparent to practitioners skilled in this art. It is intended that the scope of the invention be defined by the following claims and their equivalents.

1. A system for treating a tissue site, comprising:
at least first and second mono-polar electrodes configured to be introduced at or near a tissue site the patient; and
a voltage pulse generator coupled to the first and second mono-polar electrodes and configured to apply sufficient electrical pulses between the first and second mono-polar electrodes to induce electroporation of cells in the tissue site to create necrosis of cells of the tissue site but insufficient to create a thermal damaging effect to a majority of the tissue site.

2. The system of claim 1, further comprising:
a monitoring electrode configured to measure a test voltage delivered to cells in the tissue site.

3. The system of claim 1, wherein the test voltage is insufficient to create irreversible electroporation.

4. The system of claim 1, further comprising:
at least a third mono-polar electrode, the at least first, second and third mono-polar electrodes forming an array of electrodes.

5. The system of claim 4, wherein the array is configured to be positioned in a surrounding relationship relative to the tissue site.

6. The system of claim 1, wherein the electroporation is performed in a controlled manner with real time monitoring.

7. The system of claim 1, wherein the electroporation is performed in a controlled manner to provide for controlled pore formation in cell membrane.

8. The system of claim 1, wherein the electroporation is performed in a controlled manner to create a tissue effect in the cells at the tissue site while preserving surrounding tissue.

9. The system of claim 1, wherein the electroporation is performed in a controlled manner with monitoring of electrical impedance.

10. The system of claim 1, wherein the electroporation is monitored using an tissue imaging modality.

11. The system of claim 1, further comprising:
a device for detecting an onset of electroporation of cells at the tissue site.

12. The system of claim 11, further comprising:
a device for monitoring the effects of electroporation on cell membranes of cells at the tissue site.

13. The system of claim 11, further comprising:
a device for detecting that the effects of electroporation on cell membranes of cells at the tissue site continues.

14. The system of claim 1, wherein the electroporation is performed in a controlled manner with controlled intensity and duration of voltage.

15. The system of claim 1, wherein the electroporation is performed in a controlled manner with real time control.

16. The system of claim 1, wherein the electroporation is performed in a manner to provide for modification and control of mass transfer across cell membranes.

17. The system of claim 1, wherein the electroporation is performed in a controlled manner with a proper selection of voltage magnitude.

18. The system of claim 1, wherein the electroporation is performed in a controlled manner with a proper selection of voltage application time.

19. The system of claim 1, wherein the voltage pulse generator is configured to provide that each pulse is applied for a duration of about 5 microseconds to about 62 seconds.

20. The system of claim 1, wherein the voltage pulse generator is configured to provide that each pulse is applied for a duration of about 90 to 110 microseconds.

21. The system of claim 1, wherein the voltage pulse generator is configured to provide that each pulse is applied for a duration of about 100 microseconds.

22. The system of claim 20, wherein the voltage pulse generator is configured to apply from about 1 to 15 pulses.

23. The system of claim 20, wherein the voltage pulse generator is configured to apply about eight pulses of about 100 microseconds each in duration.

24. The system of claim 1, wherein the voltage pulse generator is configured to provide for pulse application to produce a voltage gradient at the tissue site in a range of from about 50 volt/cm to about 8000 volt/cm.

25. The system of claim 1, wherein a temperature of the tissue site is monitored and the pulses are adjusted to maintain a temperature of 100 degrees C. or less at the tissue site.

26. The system of claim 1, wherein a temperature of the tissue site is monitored and the pulses are adjusted to maintain a temperature of 75 degrees C. or less at the tissue site.

27. The system of claim 1, wherein a temperature of the tissue site is monitored and the pulses are adjusted to maintain a temperature of 60 degrees C. or less at the tissue site.

28. The system of claim 1, wherein a temperature of the tissue site is monitored and the pulses are adjusted to maintain a temperature of 50 degrees C. or less at the tissue site.

29. The system of claim 1, wherein a current-to-voltage ratio is adjusted based on temperature to maintain the tissue site temperature at 100 degrees C. or less.

30. The system of claim 1, wherein a current-to-voltage ratio is adjusted based on temperature to maintain the tissue site temperature at 75 degrees C. or less.

31. The system of claim 1, wherein a current-to-voltage ratio is adjusted based on temperature to maintain the tissue site temperature at 60 degrees C. or less.

32. The system of claim 1, wherein a current-to-voltage ratio is adjusted based on temperature to maintain the tissue site temperature at 50 degrees C. or less.

33. The system of claim 1, wherein the first electrode is placed at about 5 mm to 10 cm from the second electrode.

34. The system of claim 1, wherein the first and second mono-polar electrodes are circular in shape.

35. The system of claim 1, wherein the voltage pulse generator is configured to provide for pulse application of sufficient duration and magnitude to permanently disrupt cell membranes of cells at the tissue site.

36. The system of claim 1, wherein a ratio of electric current through cells at the tissue site to voltage across the cells is detected and a magnitude of applied voltage to the tissue site is adjusted in accordance with changes in the ratio of current to voltage.

37. A system for treating a tissue site, comprising:
a bi-polar electrode configured to be introduced at or near a tissue site of the patient; and
a voltage pulse generator coupled to the first and second electrodes and configured to apply sufficient electrical pulses to the bi-polar electrode to induce electroporation of cells in the tissue site to create necrosis of cells of the tissue site, but insufficient to create a thermal damaging effect to a majority of the tissue site.

38. The system of claim 37, further comprising:
a monitoring electrode configured to measure a test voltage delivered to cells in the tissue site.

39. The system of claim 37, wherein the test voltage is insufficient to create irreversible electroporation.

40. The system of claim 37, further comprising:
at least a second and a third bipolar electrodes, the at least first, second and third bipolar electrodes forming an array of electrodes.

41. The system of claim 40, wherein the array is configured to be positioned in a surrounding relationship relative to the tissue site.

42. The system of claim 37, wherein the electroporation is performed in a controlled manner with real time monitoring.

43. The system of claim 37, wherein the electroporation is performed in a controlled manner to provide for controlled pore formation in cell membranes.

44. The system of claim 37, wherein the electroporation is performed in a controlled manner to create a tissue effect in the cells at the tissue site while preserving surrounding tissue.

45. The system of claim 37, wherein the electroporation is performed in a controlled manner with monitoring of electrical impedance.

46. The system of claim 37, further comprising:
a device for detecting an onset of electroporation of cells at the tissue site.

47. The system of claim 37, wherein the electroporation is performed in a controlled manner with controlled intensity and duration of voltage.

48. The system of claim 37, wherein the electroporation is performed in a controlled manner with real time control.

49. The system of claim 37, wherein the electroporation is performed in a manner to for modification and control of mass transfer across cell membranes.

50. The system of claim 37, wherein the electroporation is performed in a controlled manner with a proper selection of voltage magnitude.

51. The system of claim 37, wherein the electroporation is performed in a controlled manner with a proper selection of voltage application time.

52. The system of claim 37, wherein the voltage pulse generator is configured to provide that each pulse is applied for a duration of about 5 microseconds to about 62 seconds.

53. The system of claim 37, wherein the voltage pulse generator is configured to provide that each pulse is applied for a duration of about 90 to 110 microseconds.

54. The system of claim 37, wherein the voltage pulse generator is configured to provide that each pulse is applied for a duration of about 100 microseconds.

55. The system of claim 37, wherein the voltage pulse generator is configured to apply from about 1 to 15 pulses.

56. The system of claim 53, wherein the voltage pulse generator is configured to apply about eight pulses of about 100 microseconds each in duration.

57. The system of claim 37, wherein the voltage pulse generator is configured to provide for pulse application to produce a voltage gradient at the tissue site in a range of from about 50 volt/cm to about 8000 volt/cm.

58. The system of claim 37, wherein a temperature of the tissue site is monitored and the pulses are adjusted to maintain a temperature of 100 degrees C. or less at the tissue site.

59. The system of claim 37, wherein a temperature of the tissue site is monitored and the pulses are adjusted to maintain a temperature of 75 degrees C. or less at the tissue site.

60. The system of claim 37, wherein a temperature of the tissue site is monitored and the pulses are adjusted to maintain a temperature of 60 degrees C. or less at the tissue site.

61. The system of claim 37, wherein a temperature of the tissue site is monitored and the pulses are adjusted to maintain a temperature of 50 degrees C. or less at the tissue site.

62. The system of claim 37, wherein a current-to-voltage ratio is adjusted based on temperature to maintain the tissue site temperature at 100 degrees C. or less.

63. The system of claim 37, wherein a current-to-voltage ratio is adjusted based on temperature to maintain the tissue site temperature at 75 degrees C. or less.

64. The system of claim 37, wherein a current-to-voltage ratio is adjusted based on temperature to maintain the tissue site temperature at 60 degrees C. or less.

65. The system of claim 37, wherein a current-to-voltage ratio is adjusted based on temperature to maintain the tissue site temperature at 50 degrees C. or less.

66. The system of claim 37, wherein the voltage pulse generator is configured to provide for pulse application of sufficient duration and magnitude to permanently disrupt cell membranes of cells at the tissue site.

67. The system of claim 37, wherein a ratio of electric current through cells at the tissue site to voltage across the cells is detected and a magnitude of applied voltage to the tissue site is adjusted in accordance with changes in the ratio of current to voltage.
